(19) United States
(12) Patent Application Publication
(10) Pub. No.: US 2009/0117655 A1
(43) Pub. Date: May 7, 2009

(54) Culture Matrix for Forming a Cell Spheroid, and Method of Culturing the Same

(75) Inventors: Sunao Kubota, Tokyo (JP); Yujin Kubota, Tokyo (JP); Tuyoshi Takara, Tokyo (JP)

Correspondence Address: KIRK HAHN, 14431 HOLT AVE, SANTA ANA, CA, 92705, US

(73) Assignee: TAKARA CLINIC, Tokyo (JP)

(21) Appl. No.: 11/935,354

(22) Filed: Nov. 5, 2007

(51) Int. Cl. C12N 5/00

(52) U.S. Cl. 435/397

(57) ABSTRACT

A culture method for allowing only cancer cells to grow from a cancerous organ or tissue sample removed surgically or endoscopically, forming a spheroid of cancer cells having a tissue structure and different cell stages similar to a tissue structure and cell stages in the living body, and removing the cultured spheroid of cancer cells from the matrix by changing the temperature within the physiological temperature range.

### RELATED APPLICATIONS

All publications, patents, patent applications, databases and other references cited in this application, all related applications referenced herein, and all references cited therein, are incorporated by reference in their entirety as if restated here in full and as if each individual publication, patent, patent application, database or other reference were specifically and individually indicated to be incorporated by reference.

### BACKGROUND

Advancements in gene analysis technology has made it possible to predict the therapeutic effects of anticancer drugs and radiotherapy, the possibility of metastasis, and the frequency and degree of possible side effects, which can be different between individuals receiving the same cancer therapy. These advancements are gradually making custom-made medicine a reality.

One type of genome analysis focusing on cancer is the analysis of cancer gene expression patterns using a cDNA micro-array. Although this is an innovative technology, the heterogeneity of cancerous tissue used in the expression pattern analysis makes this analysis complex and difficult, because the cancerous tissue consists of several types of normal cells, in addition to cancer cells. Since the normal cells express a specific set of genes, the cDNA micro-array analysis using traditionally collected cancerous tissue reflects a specimen of very heterogeneous cells. For this reason, there is a need in cDNA micro-array analysis to establish a culture method that separates the cancer cells from the normal cells, and grows only the cancer cells in an undamaged condition from a cancerous tissue specimen, and forming a spheroid of cancer cells having the pathological tissue structure and different cell stages similar to the tissue structure and cell stages in the living body.

The typical method used to separate the cancer cells from a cancerous organ or tissue specimen is to mechanically cut the tissue sample into thin pieces using scissors. The pieces are dispersed into individual cells using collagenase, hyaluronidase or other digestive enzymes. Next, Ficoll-Hypaque or Percoll gradients of different specific gravities are stacked, the dispersed cells are placed on top of the gradient gel, and the cancer cells are separated by centrifugation. The concentration of cancer cells after this method is about 90%. However, this means that the isolated cancer cell layer still contains around 10% normal cells. Additionally, this separation method damages the cancer cells due to the digestive enzymes used in the process.

Another method to separate cancer cells from a cancerous tissue sample is laser capture microdissection. A cap with a special film is placed on top of a frozen piece of the cancerous tissue sample stained with Hematoxylin and Eosin. Then, a person observes the sample under a microscope and the identified cancer cells are irradiated with a laser. This causes the cancer cells to attach to the cap, thus collecting the attached cancer cells.

The quantity of obtained RNA by this method is very small since the number of cancer cells that can be collected on a single glass slide from a tissue piece is limited. Therefore, the cDNA synthesized from the RNA by means of reverse transcriptase must be amplified to permit micro-array analysis. This amplification causes a difference in the ratio of each mRNA existing in the cell, and consequently the analysis does not accurately reflect the quantity of each mRNA in the cancer cell.

Another factor in using this method is that surgically removed tissue specimens are preserved in a fixative solution, which affects the mRNA. Additionally, the tissue sample only represents the stage when it was removed from the body. This method does allow the analysis of continuous gene expression patterns in chronological order.

The gene expression analysis is also affected by the stage of development of the cancer cells used in the analysis. The selection of an anti-cancer drug based on the gene expression analysis could be biased by the development stage of the cancer.

There is a need to obtain a tissue structure similar to the tissue structure in the living body. For example, different clinical results occur between cases of differentiated adenocarcinoma and cases of undifferentiated cancer, even when using the same anticancer drug. Several different anticancer drugs must be combined to treat each case of cancer for this reason. Therefore, a pathological tissue structure similar to the tissue structure in the living body must be used to make the in vitro assessment of the effectiveness of anticancer drugs.

Cell stages similar to those in a living body are needed to obtain an accurate result from cDNA micro-array analysis. Many anticancer drugs are effective only during the growth stage of the cancer cells, so the conventional two-dimensional culture method that only has a growth layer is not suitable for making a judgment on the effectiveness of the drug. The cancerous tissue sample must have the different cell stages that are similar to those in the living body. For example, the periphery must comprise a growth layer and the center must comprise dormant cells.

For a cancer cell spheroid to have cell stages similar to those in the living body, it must be 150 to 250 μm in diameter. If the cancer cell spheroid is greater than 250 μm, the center will undergo necrosis due to a lack of oxygen and nutrients.

In experimental models, the cancer growth curve follows the Gompertzian curve where the tumor doubling time increases as the tumor size increases. In the Gompertzian model, smaller tumors grow faster, so tumor regrowth between treatment cycles is more rapid when cell kill is greatest. Gompertzian growth is exponential growth with a constant exponential regression. If a tumor grows in a Gompertzian fashion, it will regress in a Gompertzian fashion.

According to the Norton-Simon theory that considers the effects of chemotherapy and radiotherapy based on the Gompertzian model, the tumor shrinking effect decreases as the tumor diameter increases (Norton, L., Semin. Oncol., 26: pp. 11-20, 1999). Therefore, a small tumor grows faster and the percentage of cells that are killed by anticancer drugs is also higher. If the tumor is too large, however, some cells with varying degrees of resistance will always exist, no matter which anticancer drug is used. The increased number of tumor cells results in tumor cells that are not exposed to anticancer drugs.

The best source of cancer cells for analysis are living cells, so methods have been developed to culture cancer cells from tissue samples. These methods include two-dimensional culture where cancer cells are cultured on the surface of a flat substratum made of glass or plastic, and three-dimensional culture where cancer cells are embedded in a matrix made of a gel or other material.

Spheroid cultures of cancer cells may better reflect characteristics of tumors than traditional monolayer cultures. Furthermore, low-passage cancer cell lines recapitulate the properties of the original tumor cells more closely than commonly used standard cell lines that experience artificial selection processes and mutations over years of passaging.

In the living body, cells exist in a three-dimensional solid structure. Therefore, it is difficult for the cells to maintain and express normal cell functions while being cultured in a two-dimensional environment. In a two-dimensional matrix, the cell contact is looser and reduces interaction between cells. Cells grown in a two-dimensional culture system do not produce cell masses (spheroid) having a three-dimensional structure or have gene expression similar to that in the living body.

In a three-dimensional matrix, cells remain in close contact with adjacent cells and therefore gene expression is controlled by the interaction between cells. Data suggests this different cell arrangement affects gene expression patterns. There is a growing interest in the development of three-dimensional culture methods that provide a rearing condition similar to the living body.

The most commonly used three-dimensional culture method is embedding cells in a gel of collagen, gelatin or Matrigel. However, this method also allows fibroblast cells to grow. Thus it is impossible to grow only cancer cells from a cancerous tissue sample using these gels. The starting cells are always contaminated with normal cells (˜10%) after centrifugation and the gel matrix does not inhibit the growth of the fibroblasts. Additionally, dissolving the gel to collect the spheroid of cancer cells is difficult and the digestive enzymes damage the spheroid of cancer cells formed in the gel. Gene analysis using these cells does not produce accurate results because of the unnecessary gene expression from the damaged cells.

Thermoreversible polymers were developed to allow removal of cells or tissues from the gel by changing the temperature. However, the gel would be liquid (sol state) at low temperatures and solid (gel state) at high temperatures. There were several problems with the initial thermoreversible polymers. The thermoreversible polymer gels required temperatures above the optimum temperature range for growing the cells, so the cells did not grow well. Additionally, the high mobility of water in these matrices was an important factor causing their degradation. The polymer structures were formed in a more open nature, indicating that water could easily attack the backbone of the polymer.

The problems with conventional thermoreversible polymer compounds having a sol-gel transition are: 1) the material may be gelled at a temperature above the sol-gel transition temperature but the gels dissolve when culture media is added, 2) the sol-gel transition temperature is higher than the body temperature (˜37° C.), and thus the materials are in the sol state at body temperature, and 3) the concentration of the macromolecular compounds in the aqueous solution must be increased considerably for effective gelation.

Newly developed thermoreversible hydrogel-forming polymer matrices and culture methods have corrected these problems. It is now possible to embed, grow and collect animal cells and tissue pieces from a three-dimensional gel based solely on a temperature change within the physiological temperature range. The thermoreversible hydrogel-forming polymer compound can be used as the culture matrix and fibroblast cells do not grow when this polymer compound is used with the culture method.

### SUMMARY OF THE INVENTION

The aim of the present disclosure is to solve the problems discovered during previous attempts to obtain only cancer cells from a cancer tissue sample, and present a method for separating cancer cells from a cancer tissue sample. The culture method forms a cancer cell spheroid having the tissue structure and different cell stages similar to the tissue structure and cell stages in a living body. The cancer cells are separated from the gel matrix by a physiological temperature change.

An extensive study was conducted and it was found that cancer cells could be separated and grown from a piece of cancerous tissue removed from a cancer patient. A spheroid of cancer cells conforming to the present disclosure is characterized by having a tissue structure and cell stages similar to the tissue structure and cell stages in the living body. Specifically, cancer cells from a cancerous organ or tissue removed surgically or endoscopically from the body and cultured according to the disclosed method will form a spheroid of cancer cells having the tissue structure similar to the tissue structure in the living body. Also, the periphery of the spheroid of cancer cells exhibits the characteristics of a growth layer while the center exhibits the characteristics of a dormant layer. The spheroid of cancer cells is classified by its exhibition of characteristics: highly differentiated adenocarcinoma, moderately differentiated adenocarcinoma, poorly differentiated adenocarcinoma, undifferentiated cancer or unclassifiable cancer.

The disclosure relates to a culture matrix and method that allows only cancer cells to grow from an organ or tissue with cancer removed surgically or endoscopically. The cells form a spheroid of cancer cells that have the pathological tissue structure and cell stages similar to the tissue structure and cell stages in the living body.

The disclosed matrix is a thermoreversible hydrogel-forming polymer in which the sol-gel transition phase is reversibly changed by temperature in a three-dimensional culture system. Human cancer cells form multicellular spheroids within the matrix. This matrix has several advantages over previously used matrices for culturing cells. First, cancer cells grow three-dimensionally with this polymer; second, it is easy to harvest cells or spheroids from this polymer by simply cooling down the temperature; and third, the matrix is translucent so the cells or spheroids can be observed through a phase-contrast microscope. Thus, this matrix is a very useful material for three-dimensional cultures.

An aqueous solution of thermoreversible hydrogel-forming polymer is a fluid below the reversibly, sol-gel transition temperature, which turns into an elastic hydrogel above the sol-gel transition temperature. Using this feature, a three dimensional culture system is possible that enables cells to be embedded and recovered by just changing the temperature and without using enzymes.

The culture matrix according to the present disclosure has a function of inhibiting or suppressing the excessive growth of fibroblasts.

The culture matrix according to the present disclosure assumes a sol state having a fluidity at a low temperature, and therefore, the cell or tissue-culturing matrix can easily be mixed or inoculated with a cell or tissue from a living organism.

The culture matrix according to the present disclosure can be converted into a gel state and the cancer cells can be cultured three-dimensionally in the same manner as in a living body.

When the culture matrix is cooled after the culture of the cell or tissue, the matrix returns to a liquid sol state at a low temperature. Thus, the cells can be easily recovered and they are not damaged by excessive temperature.

### BRIEF DESCRIPTION OF THE DRAWINGS

FIG. 1 is a microphotograph showing cells growing from periphery of cancer tissue specimen.

FIG. 2 is a microphotograph showing a mass of cells.

FIG. 3 is a microphotograph showing cells breaking away from the tissue specimen and forming colonies.

FIG. 4 is a microphotograph showing cells of 30 to 50 μm on day 3.

FIG. 5 is a microphotograph showing ˜100 μm on day 7.

FIG. 6 is a microphotograph showing ˜200 μm on day 14.

FIG. 7 is a microphotograph showing ˜500 μm on day 28.

FIG. 8 is a microphotograph showing cells stained with Hematoxylin and Eosin exhibiting characteristics of moderately differentiated adenocarcinoma.

FIG. 9 is a microphotograph showing cells stained with BrdU indicating a darkly stained growth layer and a dormant layer in the center.

### DETAILED DESCRIPTION

A tumor spheroid with a diameter of 50 to 100 μm has a short tumor doubling time, while one with a diameter of 250 μm or more undergoes necrosis at the center of the spheroid. Accordingly, an ideal size of cancer cell spheroid is 100 to 200 μm.

The optimal sol-gel transition temperature for the matrix used in the present method should preferably have a sol-gel transition temperature of above 0° C. but not exceeding 42° C. In particular, a temperature range of 4° C. or above, but not exceeding 40° C. is ideal. This facilitates the separation of cancer cells from the excised cancerous tissue and prevention of heat damage to the cancer cells in the spheroid.

An ideal structure of the thermoreversible hydrogel-forming polymer compound is that the compound comprises multiple blocks having a cloud point that are bonded with a hydrophilic block. Presence of the multiple blocks having a cloud point is preferred because they allow the compound to become a gel state at temperatures higher than the hydrogel transition temperature.

The presence of the hydrophilic block allows the hydrogel to become water-soluble at temperatures lower than the sol-gel transition temperature. Since the blocks having a cloud point dissolve in water at temperatures lower than the cloud point, while turning water-insoluble at temperatures higher than the cloud point, these blocks serve as cross-linking points constituted by hydrophobic bonds to form a gel. The cloud point associated with the occurrence of hydrophobic bonding corresponds to the sol-gel transition temperature of the hydrogel.

The cloud point can be measured by, for example, cooling an aqueous solution containing the polymer compound (˜1 percent by weight) so that the aqueous solution becomes a clear solution, and then gradually raising the temperature of the solution at a rate of ˜1° C. per minute to find the point at which the solution starts to become cloudy. This temperature defines the cloud point.

The hydrophilic block in the aforementioned thermoreversible hydrogel-forming polymer has the function to turn the thermoreversible hydrogel-forming polymer water-soluble at temperatures lower than the sol-gel transition temperature. At the same time, it also has the function, at temperatures higher than the transition temperature, to prevent the hydrogel from depositing and precipitating due to an excessive hydrophobic bonding power, and thereby allowing a hydrous gel state to form.

The blocks having a cloud point are selected from among temperature-sensitive polymers, whose representative examples include poly N-substituted acrylic amide derivatives, poly N-substituted methacryl amide derivatives, partially acetylated polyvinyl alcohols and polyvinyl methyl ethers. Temperature-sensitive polymer compounds (Heskins M. and Guillet J. E., J. Macromol. Sci., A2 (8), 1441 (1968)) are polymer compounds whose solubility temperature coefficient to water is negative, and which contain nonpolar groups (isopropyl groups) that turn hydrophobic as the temperature rises and thereby cause the polymer compound to deposit and precipitate due to hydrophobic bonding occurring among molecules, while also containing polar groups (amide groups) that turn hydrophilic as the temperature drops and consequently allow the polymer compound to dissolve in water.

Examples of the hydrophilic block that bonds with the aforementioned blocks having a cloud point include, among others, polyethylene oxide, polyvinyl alcohol, polyacryl amide, polymethacryl amide, polyhydroxy methyl acrylate, polyacrylic acid, and polymethacrylic acid.

A thermoreversible hydrogel-forming polymer compound in which multiple blocks having a cloud point are bonding with a hydrophilic block completely dissolves in water and assumes a sol state at temperatures lower than the cloud point, because the “blocks having a cloud point” in the molecule are water-soluble, just like the hydrophilic block. On the other hand, raising the temperature of an aqueous solution of this thermoreversible hydrogel-forming polymer to temperatures higher than the aforementioned cloud point causes the “blocks having a cloud point” in the molecule to turn hydrophobic, thus triggering a hydrophobic interaction to cause the thermoreversible hydrogel-forming polymer to associate with other different molecules.

At this time, the hydrophilic block remains water-soluble (even after the aqueous solution has been heated to temperatures higher than the cloud point), and this causes the polymer to form, in the aqueous solution, a hydrogel having a three-dimensional net-like structure constituted by the blocks having a cloud point that are cross-linked by hydrophobic associations. When the temperature of this hydrogel is lowered again to levels lower than the cloud point of the “blocks having a cloud point” in the molecule, the blocks having a cloud point turn water-soluble, thereby freeing the cross-linking points due to hydrophobic association. As a result, the hydrogel structure disappears and the polymer becomes a complete aqueous solution again.

The solubility of the gel in the gel state is very low. An aqueous solution of the thermoreversible hydrogel-forming polymer compound proposed by the present disclosure has such property that, when the aqueous solution is gelled at a temperature higher than the sol-gel transition temperature and then the obtained gel is soaked in water at a volume ratio of approximately 0.1 to 10 times, the gel will not dissolve in water for a long period of time. This property of the polymer used in the present disclosure is realized by, for example, the presence in the polymer of multiple blocks having a cloud point.

A tumor has most of its cells in S phase (replication stage) and the cells are more active than normal cells, since most normal cells are in the G0 stage. Groups of cancer cells will have specific cell stages and grow through repeated cell divisions. These cell groups do not have uniform cell stages, because cells divide more slowly at the center of the solid cancer mass and other locations that do not receive a sufficient supply of oxygen and other substances needed for cells to survive. Some of these cells remain in the so-called dormant stage (G0 stage) by deviating from the division cycle.

The effect of a chemotherapy is determined by the cell stages present in the tumor. Antitumor drugs are classified by which cell stage is targeted or whether it is not specific to a particular cell stage. Drugs that target cells at a specific stage are generally more effective on tumors undergoing active cell division; however, they are less effective on cells in the dormant stage. This is the reason why a single administration of a chemotherapy is not sufficient to kill all the tumor cells, and a normal clinical practice is to administer these drugs continuously or in cycles.

The matrix disclosed in the present application has a transition temperature lower than previously described thermoreversible hydrogel-forming polymers. Its properties allow for better culture conditions and the cells grow into a spheroid that mimics natural tissue in a living animal. The individual cancer cells and spheroid cell masses are able to grow in the disclosed matrix with the disclosed culture methods to produce cells with the same characteristics as found in the excised cancer sample.

The present application uses the matrix to aid in the culture of cancer cells without the hindrance of fibroblast cells. The matrix allows only the cancer cells in the tissue specimen to grow and multiply when cultured in the gelled state.

The matrix and culture method uses only physiological temperatures (0° C. to ˜40° C.) to grow and separate the cancer cell from the specimen and, at the same time, the cell mass resembles in vivo growth (spheroid). The matrix and culture method is the main reason the cancer cells are separated from the other cells.

There are major differences between the disclosed matrix and previously disclosed thermoreversible hydrogel-forming polymer matrices. In the present application, the cancer cells that grow from the excised cancerous tissue specimen are able to be separated from the other cell types while only exposing the cells to physiological temperature variations. Additionally, the isolated cancer cells are similar in form to natural cells and the spheroid cell mass shows a natural cell cycle.

The cell mass of growing cancer cells from a cancerous tissue specimen grown in the previous thermoreversible hydrogel-forming polymer matrices had fibroblasts present, which was a troublesome characteristic of these previous culture methods.

Also, the disclosed matrix allows the use of culture media that would cause previous thermoreversible hydrogel-forming polymer matrices to dissolve.

Therefore, in a strict sense these thermoreversible hydrogel-forming polymer matrices do not possess a hydrophobic part and a hydrophilic part inside the molecule. When the culture solution is placed on the gel of previous thermoreversible hydrogel-forming polymer matrices on the 3^{rd} or 4^{th} day; the bonding decreases, causing the gel to become more liquid and melting the gel. The reason the gel melts is because there are few hydrophobic parts and the gel absorbs the culture solution. It is believed that the hydrophobic bonds between the molecules break due to the swelling.

The disclosed matrix has different characteristics from an aqueous solution of hydrogel. After gelling, at a temperature higher than the sol-gel transition temperature, a piece of gel (specific volume=0.1) in a solution that is 50 times the volume of the gel will not melt even after an extended period of time.

The matrix disclosed in the present application has many non-polarized regions and an increase in temperature causes a change in the hydrophilic block, and hydrophobic bonds form. The three-dimensional network structure is finer than in comparison to previous thermoreversible hydrogel-forming polymer matrices. The absorption of the culture medium with the disclosed matrix is very low so there is very little swelling and thus no breaking of the hydrophobic bonds.

The sol-gel transition temperature of the disclosed matrix is at 40° C. (clouding point) so when the matrix temperature is lowered towards 0° C. to liquify the matrix, the cancer cells do not get thermal damage due to consecutive operations during the culture method.

An important advantage of the disclosed matrix is that it allows the culture medium to be replaced by simply changing the medium covering the pieces of matrix. The nutrients in the culture medium permeate through the matrix to the cancer cells inside the gel and conversely, the metabolic wastes permeate out of the gel. This allows fresh culture media to be used without disassociating the gel.

It was impossible to produce spheroids of cancer cells without fibroblast cells using previously disclosed thermoreversible hydrogel-forming polymer matrices because the culture medium would cause the gel to “melt” after 3 to 4 days. Then, the mass of cancer cells would come in contact with the substratum of the culture container and the fibroblast cells would multiply.

### DEFINITIONS

### Cancerous Tissues or Cancerous Cells

Cancerous tissues or cancerous cells described in this disclosure refer to tissues containing cancer cells collected directly or surgically removed from an animal or human. Although the source organ is not limited, examples include stomach, esophagus, small intestines, large intestines, liver, pancreas, uterus, ovary, bone, and brain.

### Culture Medium

Any known cell culture medium can be used without limitation as the medium to be used in combination with the sol-gel polymer compounds.

### Anticancer Drugs

Anticancer drugs can include any general anticancer drugs currently used in cancer therapy, as well as, new anticancer drugs to be developed in the future.

### Sol State

1 ml of a hydrogel in a sol state is poured into a test tube having an inside diameter of 1 cm, and is left standing for 12 hours in a water bath which is controlled at a predetermined temperature (constant temperature). Thereafter, when the test tube is turned upside down, if the interface (meniscus) between the solution and air is deformed (inclusive a case wherein the solution flows out from the test tube) due to the weight of the solution, the above polymer solution is defined in a “sol state” at the above-mentioned predetermined temperature.

### Gel State

1 ml of a hydrogel in a sol state is poured into a test tube having an inside diameter of 1 cm, and is left standing for 12 hours in a water bath which is controlled at a predetermined temperature (constant temperature). If the interface (meniscus) between the solution and air is not deformed due to the weight of the solution, even when the test tube is turned upside down, the above polymer solution is defined in a “gel state” at the above-mentioned predetermined temperature.

### Sol-Gel Transition Temperature

The temperature of a hydrogel in a “sol state” (solution) (e.g., 1% (wt)) is gradually increased (e.g., in 1° C. increment) until the hydrogel converts to the “gel state” (solid). Conversely, the temperature of a hydrogel in the “gel state” is gradually decreased (e.g., in 1° C. increment) until the hydrogel converts to the “sol state”. This is defined as the “sol-gel transition temperature”.

### Cell Stages

All cells grow with a life cycle of five stages, repeated during each doubling. The cell stages are G1 stage (interphase stage), S phase (replication stage), G2 stage (interphase stage), M stage (division stage), and then back to G1 stage. Control of the cell stages is vital to continued life. The G1 stage varies in duration, but it normally lasts 4 to 24 hours. If this stage becomes prolonged the cells normally enter the G0 phase, or dormant stage. The S stage is when DNA is synthesized and normally lasts 10 to 20 hours. The G2 stage is the stage before cell division and lasts 2 to 10 hours. The actual duration of the M stage is 0.5 to 1 hour.

### Cell Spheroid

A spheroid of cancer cells is a cell mass of spherical shape cultured in vitro, consisting of multiple layers of cancer cells or established cancer cells separated/collected directly from an animal. Spheroids consisting of normal cells, such as liver cells, have morphology and functions similar to those in the living body (Takezawa, T. et al, J Cell Sci, 101, pp. 495-501, 1992).

### EMBODIMENTS

In an embodiment of using the matrix, a cell or tissue containing the cell is first inoculated or mixed into the matrix according to the present disclosure. In order to carry out such inoculation or mixing, for example, a thermoreversible hydrogel-forming polymer constituting the matrix used for culturing a cell or tissue of the present disclosure is dissolved in a culture medium such as RPMI-1640 at a low temperature (e.g., 4° C.) while stirring, so that the matrix according to the present disclosure is converted into a state of an aqueous solution (a sol state) with a temperature lower than its sol-gel transition temperature, and then the above cell or tissue may be added or suspended therein. The culture medium used herein is not limited. A culture medium in which a cell of interest (for example, but not limited to, any cell from a living animal, embryonic cell, embryonic stem cell, adult stem cell, engineered cell, cancer cell, cell line) easily grows or differentiates may be appropriately selected and used. In addition, any chemical mediator promoting the growth or differentiation of the cell of interest may also be added to the culture medium.

An embodiment of the present disclosure is to provide a cell or tissue-culturing matrix which functions as a thermoreversible hydrogel-forming polymer, and a method of culturing a cell or tissue using the matrix.

An embodiment of the present disclosure is to provide a cell or tissue-culturing matrix which can effectively regenerate an intended cell or tissue, while suppressing growth of fibroblasts, and a method of culturing a cell or tissue by using the matrix.

An embodiment of the present disclosure is to provide a cell or tissue-culturing matrix which enables the inoculation or mixing of various cells or tissue containing these cells, and can function as adhesion, differentiation or morphogenesis of various cells so as to culture the tissue or organ, and a method of culturing a cell or tissue by using the matrix.

An embodiment of the present disclosure is a thermoreversible hydrogel-forming polymer containing water or any appropriate culture medium.

An embodiment of the present disclosure is a thermoreversible hydrogel-forming polymer containing a chemical mediator, which promotes the regeneration of a tissue or organ in a living organism.

An embodiment of the present disclosure is a thermoreversible hydrogel-forming polymer that includes multiple blocks having a cloud point, and a hydrophilic block connected or combined therewith.

An embodiment of the present disclosure is a thermoreversible hydrogel-forming polymer with a sol-gel transition temperature between 0° C. and 42° C. such that it assumes a sol state at a low temperature and assumes a gel state at a higher temperature.

An embodiment of the present disclosure is a thermoreversible hydrogel-forming polymer with a sol-gel transition temperature between 4° C. and 40° C. An embodiment of the present disclosure is a thermoreversible hydrogel-forming polymer with a sol-gel transition temperature at 0° C., 1° C., 2° C., 3° C., 4° C., 5° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., or 42° C. An embodiment of the present disclosure is a thermoreversible hydrogel-forming polymer with a sol-gel transition temperature within a range of temperatures between any temperature between 0° C. and 42° C., and a higher temperature within the range of 0° C. to 42° C.

An embodiment of the present disclosure is a thermoreversible hydrogel-forming polymer culture matrix comprising multiple blocks having a cloud point bonded with a hydrophilic block and a sol-gel transition temperature between 0° C. and 42° C.

An embodiment of the present disclosure is a thermoreversible hydrogel-forming polymer culture matrix where the multiple blocks are selected from the group consisting of poly N-substituted acrylic amide derivatives, poly N-substituted methacryl amide derivatives, partially acetylated polyvinyl alcohols and polyvinyl methyl ethers.

An embodiment of the present disclosure is a thermoreversible hydrogel-forming polymer culture matrix where the hydrophilic block is selected from the group consisting of polyethylene oxide, polyvinyl alcohol, polyacryl amide, polymethacryl amide, polyhydroxy methyl acrylate, polyacrylic acid, and polymethacrylic acid.

An embodiment of the present disclosure is a thermoreversible hydrogel-forming polymer culture matrix where the culture matrix is adapted to inhibit fibroblast growth.

An embodiment of the present disclosure is a thermoreversible hydrogel-forming polymer culture matrix where the culture matrix is adapted to form a spheroid of cells.

An embodiment of the present disclosure is a thermoreversible hydrogel-forming polymer culture matrix where the spheroid of cells has the characteristics of cancer cells in the living body.

An embodiment of the present disclosure is a thermoreversible hydrogel-forming polymer culture matrix where the matrix in the gel state is insoluble in a liquid at a volume ratio of approximately 0.1 to 10 times the volume of the gel.

An embodiment of the present disclosure is a culture method to form spheroid of cells and inhibit fibroblast cells comprising: placing a tissue sample in a solution of a thermoreversible hydrogel-forming polymer, with a sol-gel transition temperature between 0° C. and 42° C., at a temperature below the sol-gel transition temperature, raising the temperature of the solution of the thermoreversible hydrogel-forming polymer above the sol-gel transition temperature, forming the solution of the thermoreversible hydrogel-forming polymer to a gel state, and culturing the tissue sample in the gelled thermoreversible hydrogel-forming polymer with an appropriate culture medium and under an appropriate culture condition for the tissue sample.

An embodiment of the present disclosure is a culture method to form spheroid of cells and inhibit fibroblast cells further comprising: cooling the temperature of the gelled thermoreversible hydrogel-forming polymer containing the tissue sample below the sol-gel transition temperature, allowing the thermoreversible hydrogel-forming polymer to become the sol state, and removing newly grown spheroids of cells.

An embodiment of the present disclosure is a culture method to form spheroid of cells and inhibit fibroblast cells where the newly grown spheroid of cells is placed in a new solution of the thermoreversible hydrogel-forming polymer for continued culture.

An embodiment of the present disclosure is a culture method to form spheroid of cells and inhibit fibroblast cells where the newly grown spheroid of cells is used for gene analysis.

An embodiment of the present disclosure is a culture method to form spheroid of cells and inhibit fibroblast cells where the newly grown spheroid of cells have the structure characteristics of tissue in a living animal.

An embodiment of the present disclosure is a culture method to form spheroid of cells and inhibit fibroblast cells where the solution of the thermoreversible hydrogel-forming polymer becomes a gel state at a temperature above the sol-gel transition temperature and the gel state is insoluble in a liquid at a volume ratio of approximately 0.1 to 10 times the volume of the gel.

An embodiment of the present disclosure is a culture method to form spheroid of cells and inhibit fibroblast cells where the culture medium is changed with new culture medium and the thermoreversible hydrogel-forming polymer remains in the gelled state.

An embodiment of the present disclosure is a culture method to form spheroid of cells and inhibit fibroblast cells where the tissue sample is an aggregation of cells.

An embodiment of the present disclosure is a culture method to form spheroid of cells and inhibit fibroblast cells where the aggregation of cells is an aggregation of endocrine cells.

An embodiment of the present disclosure is a culture method to form spheroid of cells and inhibit fibroblast cells where the aggregation of endocrine cells is an islet.

An embodiment of the present disclosure is a method of culturing a cell or tissue with a matrix of thermoreversible hydrogel-forming polymer having a sol-gel transition temperature such that it assumes a sol state at a low temperature and assumes a gel state at a higher temperature

An embodiment of the present disclosure is a method of culturing a cell or tissue with a matrix of thermoreversible hydrogel-forming polymer that inhibits the growth of fibroblast cells.

An embodiment of the present disclosure is a method of culturing a cell or tissue at a temperature within the physiological temperature range in a matrix of thermoreversible hydrogel-forming polymer with a sol-gel transition temperature between 0° C. and 42° C. such that the physiological temperature is higher than the sol-gel transition temperature.

An embodiment of the present disclosure is a method of culturing a cell or tissue with a matrix of thermoreversible hydrogel-forming polymer that includes multiple blocks having a cloud point, and a hydrophilic block connected or combined therewith.

An embodiment of the present disclosure is a method of culturing a cell or tissue with a matrix of thermoreversible hydrogel-forming polymer with a chemical mediator, which promotes the regeneration of a tissue or organ in a living organism.

An embodiment of the present disclosure is a method of culturing a cell or tissue with a matrix of thermoreversible hydrogel-forming polymer that is water insoluble in the gel state.

An embodiment of the present disclosure is a method of culturing a cell or tissue with a matrix of thermoreversible hydrogel-forming polymer and any appropriate culture medium.

An embodiment of the present disclosure is a method of culturing a cell or tissue with a matrix of thermoreversible hydrogel-forming polymer and any appropriate culture procedure.

Embodiments of the present disclosure are not limited by the above listed embodiments. It is to be understood that while the invention has been described in conjunction with the detailed description thereof, the foregoing descriptions are intended to illustrate and not limit the scope of the invention, which is defined by the scope of the appended claims. For example, although the above descriptions relate to human cancer cells, various aspects of the invention might also be applied to cells from other animals (e.g., mammals, avians, fishes, crustaceans, and domestic and farm animals) and any cell from a living animal, embryonic cell, embryonic stem cell, adult stem cell, engineered cell, cancer cell, cell line or other cell types by making appropriate modifications to the described methods.

### EXAMPLES

The disclosed method is described with more specificity in the following examples. It should be noted, however, that the scope of the disclosed method is not limited by the examples.

### Experiment 1

### Sol-Gel

N-isopropylacrylamide (NIPAAm) [59.2 g] and butylmethacrylate (BMA) (Wako) [3.04 g] were melted in ether [606.4 g] and distilled water [257.2 g] was added. A 20% aqueous solution of polyethylene glycol was made by adding 124.4 g (PDE6K) (NOF Corporation) to 160 ml of distilled water and heating to 70° C. under nitrogen. Four milliliters of 10% persulfate ammonium (Wako) and 0.4 ml of tetramethylethylenediamine (Wako) were added every 30 minutes (6 times) and the polyethylene glycol was polymerized. After cooling, it was diluted in 6 liters of distilled water. The solution was concentrated to approximately 1 liter by using a hollow fiber ultrafiltration membrane with a molecular weight cutoff of 100,000 (Amicon, HIP100-43) at a temperature below 10° C. The dilution and concentration process was repeated 6 times. The final concentrated liquid hydrogel was freeze-dried.

### Example 2

### Experiment to Separate and Grow Only Cancer Cells from a Cancerous Tissue

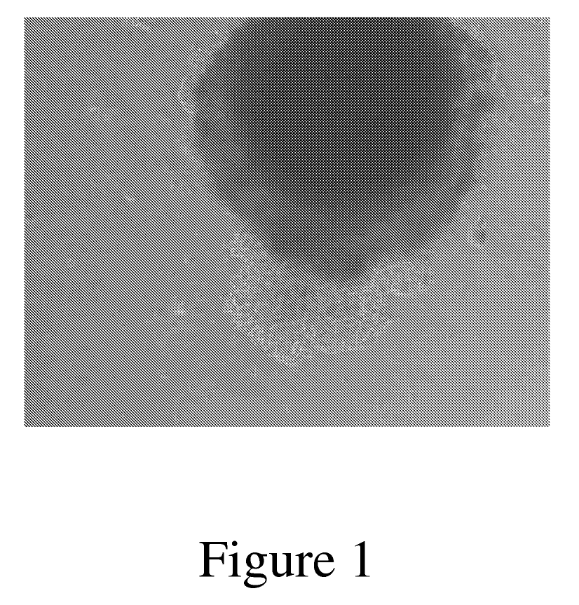

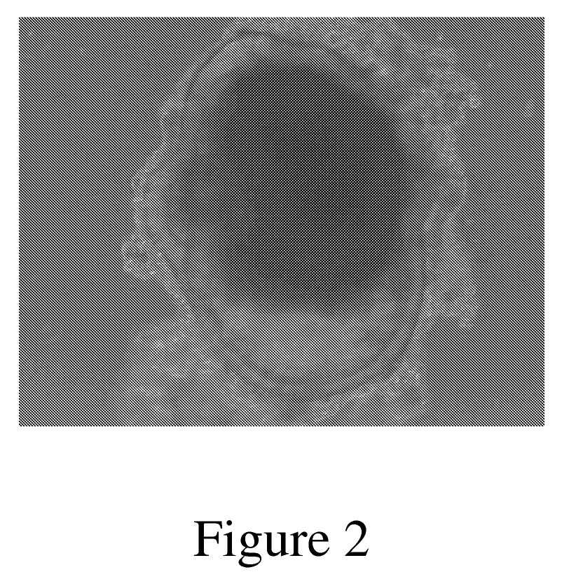

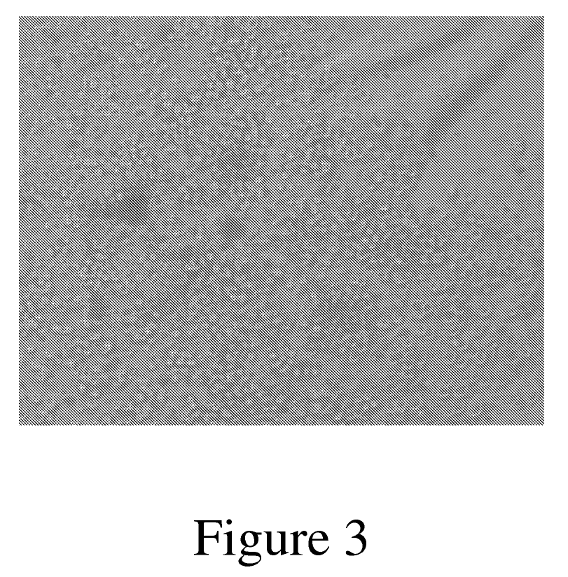

A cancerous tissue of 1.0 cm³ in volume was surgically removed from the large intestines of a colon cancer patient. The cancerous tissue was cut into 0.5 mm squares using a tissue chopper. On a hot plate preheated to 37° C., 200 μl of cooled RPMI-1640 culture medium containing 8% thermoreversible hydrogel-forming polymer was injected into a 24-well plate. The RPMI-1640 containing the matrix immediately changed into a gel. Two to three cut tissue pieces were immersed in the gel. A solution was prepared by adding 10% fetal calf serum, 100 μg/ml of penicillin, 100 μg/ml of streptomycin, 50 μg/ml of amphotericin and 5 μg/ml of insulin to the RPMI-1640 culture solution, and 300 μl of the prepared solution was added to the gel in each well. The gel was then cultured in an atmospheric condition of 37° C. and 5% CO₂. The form of the tissue pieces in the gel was observed during culture with a phase contrast microscope. After two to three days in culture, the cells grew in a germination-like manner from the periphery of cancerous tissue pieces (FIG. 1). These cells had grown to a size of ˜50 μm and formed a mass by day 5 to 7 (FIG. 2). Further culture caused cells to break away from the tissue pieces to form colonies (FIG. 3). Fibroblast growth from the cancerous tissue pieces was not observed.

The 24-well plate was placed on ice to cause the gel to change to water-soluble sol, so only grown cancer cells are separated and collected from tissue pieces in the matrix gel. When a tissue piece in water-soluble sol was gently shaken with tweezers, a cell mass separated from the tissue piece. The tissue pieces were removed and the thermoreversible hydrogel-forming polymer solution containing the cancer cells was placed in a 10 ml centrifugal tube, mixed with 5 ml of RPMI-1640 culture medium, and then the mixture was centrifuged at 500 G for 3 minutes to collect the cancer cells.

### Example 3

### Method to Prepare a Spheroid of Cancer Cells Constituted Only by Cancer Cells

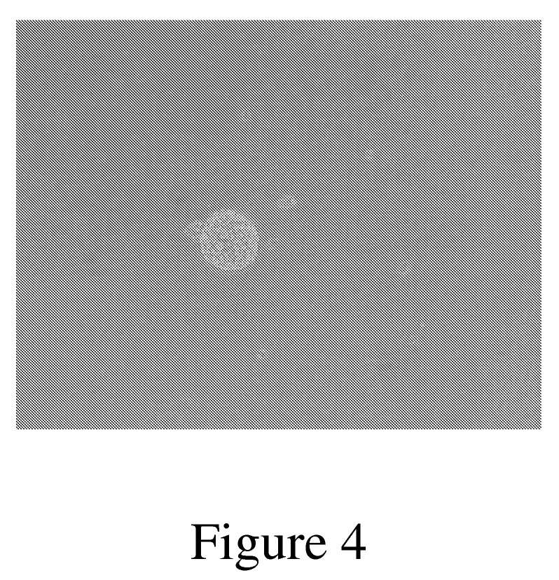

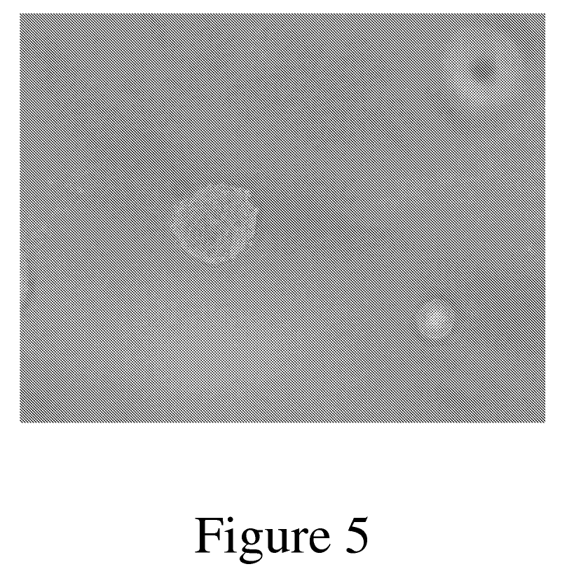

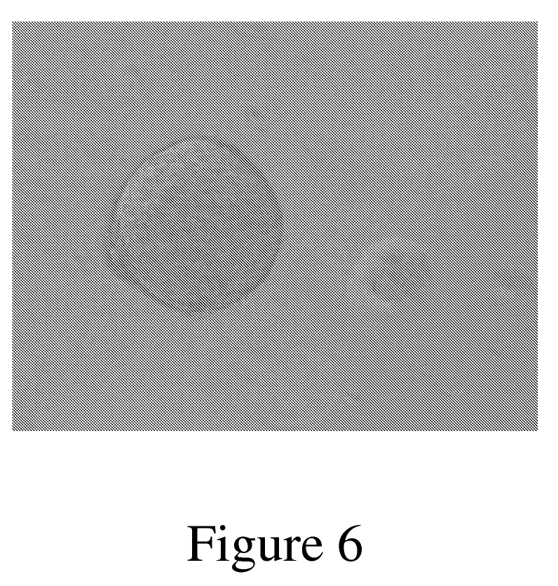

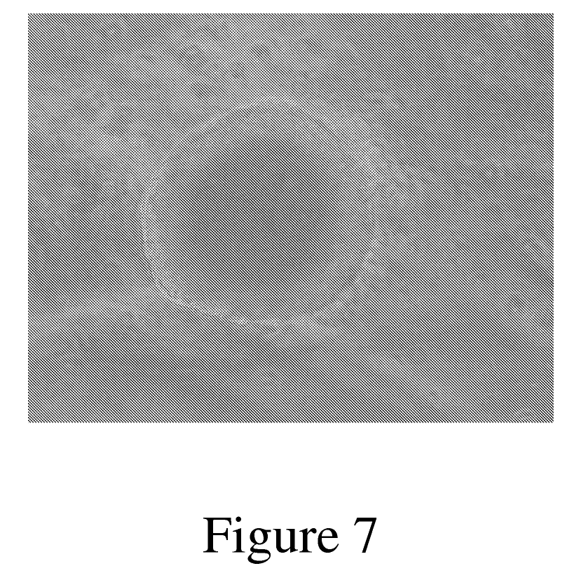

Cancer cells separated/collected from a tissue piece was suspended in cooled RPMI-1640 solution containing 8% thermoreversible hydrogel-forming polymer so that the cancer cell content became 1×10⁴ cells/ml, and 1 ml of the suspension was placed onto a culture plate with a diameter of 35 mm (Falcon 3001 manufactured by Nippon Becton Dickinson Company, Ltd.). The culture plate was placed on a hot plate preheated to 37° C. to change the thermoreversible hydrogel-forming polymer solution into gel completely. Then, 300 μl of a solution prepared by adding 10% fetal calf serum, 100 μg/ml of penicillin, 100 μg/ml of streptomycin, 50 μg/ml of amphotericin and 5 μg/ml of insulin to culture solution RPMI-1640 was added to the gel in each well. The gel was then cultured in an atmospheric condition of 37° C. and 5% CO₂. The form of tissue pieces in the gel was observed daily using a phase difference microscope. The microphotographs are shown. As evident from these microphotographs, cells grew daily in the gel and their size increased to 30 to 50 μm on day 3 (FIG. 4), ˜100 μm on day 7 (FIG. 5), ˜200 μm on day 14 (FIG. 6), ˜300 μm on day 21, and ˜500 μm on day 28 (FIG. 7).

On day 14, to collect spheroids of cancer cells with a size of ˜200 μm, a culture plate with a diameter of 35 mm was placed on ice to change the gel into sol completely. As a result, spheroids of cancer cells could be collected in undamaged condition and without the spheroid dispersing into cells.

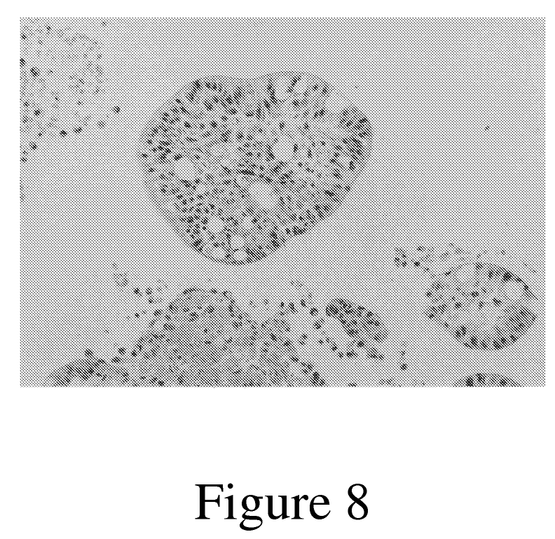

Hematoxylin and Eosin staining of these spheroids of cancer cells found that the spheroids had the same pathological form as a surgically removed colon cancerous tissue, and they also exhibited the characteristics of moderately differentiated adenocarcinoma (FIG. 8).

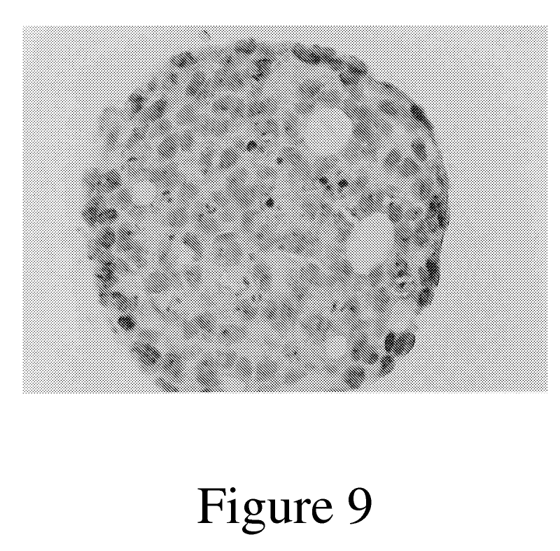

When the metabolically active areas of these spheroid of cancer cells were examined using the BrdU staining method, the darkly stained surface layer was found to constitute a growth layer, while the center was found to be a dormant layer (FIG. 9).

As explained above, the present disclosure has made it possible to separate only cancer cells from a cancerous tissue collected directly from a cancer patient and to form a spheroid of cancer cells in a simple manner based on temperature change within the physiological temperature range and without damaging the cancer cells. By using a spheroid of cancer cells obtained in accordance with the present disclosure, it is possible to select anticancer drugs effective in treating the cancer of a given cancer patient, and predict level of resistance. Additionally, it can predict the metastatic capability of the cancer cells and organs to which they will metastasize.

What is claimed is:

1. A thermoreversible hydrogel-forming polymer culture matrix comprising multiple blocks having a cloud point bonded with a hydrophilic block, and a sol-gel transition temperature between 0° C. and 42° C.

2. The thermoreversible hydrogel-forming polymer culture matrix of claim 1, where the multiple blocks are selected from the group consisting of poly N-substituted acrylic amide derivatives, poly N-substituted methacryl amide derivatives, partially acetylated polyvinyl alcohols and polyvinyl methyl ethers.

3. The thermoreversible hydrogel-forming polymer culture matrix of claim 1, where the hydrophilic block is selected from the group consisting of polyethylene oxide, polyvinyl alcohol, polyacryl amide, polymethacryl amide, polyhydroxy methyl acrylate, polyacrylic acid, and polymethacrylic acid.

4. The thermoreversible hydrogel-forming polymer culture matrix of claim 1, where the sol-gel transition temperature is between 4° C. and 40° C.

5. The thermoreversible hydrogel-forming polymer culture matrix of claim 1, where the culture matrix is adapted to inhibit fibroblast growth.

6. The thermoreversible hydrogel-forming polymer culture matrix of claim 1, where the culture matrix is adapted to form a spheroid of cells.

7. The thermoreversible hydrogel-forming polymer culture matrix of claim 6, where the spheroid of cells has the characteristics of cancer cells in the living body.

8. The thermoreversible hydrogel-forming polymer culture matrix of claim 1, where the matrix in the gel state is insoluble in a liquid at a volume ratio of approximately 0.1 to 10 times the volume of the gel.

9. A culture method to form a spheroid of cells and inhibit fibroblast cells comprising:
placing a tissue sample in a solution of a thermoreversible hydrogel-forming polymer, with a sol-gel transition temperature between 0° C. and 42° C., at a temperature below the sol-gel transition temperature,
raising the temperature of the solution of the thermoreversible hydrogel-forming polymer above the sol-gel transition temperature,
forming the solution of the thermoreversible hydrogel-forming polymer to a gel state, and
culturing the tissue sample in the gelled thermoreversible hydrogel-forming polymer with an appropriate culture medium and under an appropriate culture condition for the tissue sample.

10. The culture method to form a spheroid of cells and inhibit fibroblast cells of claim 9, further comprising:
cooling the temperature of the gelled thermoreversible hydrogel-forming polymer containing the tissue sample below the sol-gel transition temperature,
allowing the thermoreversible hydrogel-forming polymer to become the sol state, and
removing newly grown spheroids of cells.

11. The culture method to form a spheroid of cells and inhibit fibroblast cells of claim 9, where the newly grown spheroid of cells is placed in a new solution of the thermoreversible hydrogel-forming polymer for continued culture.

12. The culture method to form a spheroid of cells and inhibit fibroblast cells of claim 9, where the newly grown spheroid of cells is used for gene analysis.

13. The culture method to form a spheroid of cells and inhibit fibroblast cells of claim 9, where the newly grown spheroid of cells has the structure characteristics of tissue in a living animal.

14. The culture method to form a spheroid of cells and inhibit fibroblast cells of claim 9, where the solution of the thermoreversible hydrogel-forming polymer becomes a gel state at a temperature above the sol-gel transition temperature and the gel state is insoluble in a liquid at a volume ratio of approximately 0.1 to 10 times the volume of the gel.

15. The culture method to form a spheroid of cells and inhibit fibroblast cells of claim 9, where the culture medium is changed with new culture medium, and the thermoreversible hydrogel-forming polymer remains in the gelled state.

16. The culture method to form a spheroid of cells and inhibit fibroblast cells of claim 9, where the tissue sample is an aggregation of cells.

17. The culture method to form a spheroid of cells and inhibit fibroblast cells of claim 16, where the aggregation of cells is an aggregation of endocrine cells.

18. The culture method to form a spheroid of cells and inhibit fibroblast cells of claim 17, where the aggregation of endocrine cells is an islet.
